(19) United States
(12) Patent Application Publication
(10) Pub. No.: US 2020/0347393 A1
(43) Pub. Date: Nov. 5, 2020

(54) MICRO- AND NANONEEDLES FOR PLANT AND OTHER CELL PENETRATION

(71) Applicant: President and Fellows of Harvard College, Cambridge, MA (US)

(72) Inventors: Hongkun Park, Cambridge, MA (US); Xing Liao, Cambridge, MA (US); Jeffrey T. Abott, Cambridge, MA (US); Steven Ban, Cambridge, MA (US); Tianyang Ye, Cambridge, MA (US)

(73) Assignee: President and Fellows of Harvard College, Cambridge, MA (US)

(21) Appl. No.: 16/770,012

(22) PCT Filed: Dec. 7, 2018
(86) PCT No.: PCT/US2018/064372

§ 371 Date: Jun. 4, 2020

Related U.S. Application Data
(60) Provisional application No. 62/596,533, filed on Dec. 8, 2017.

(51) Int. Cl. C12N 15/82

(52) U.S. Cl. CPC C12N 15/8207

(57) ABSTRACT

The present invention generally relates to micro- and nanoneedles, e.g., for use in penetrating plant cells, or other cells. In some embodiments, delivery of target biomolecules into cells with protective outer layers (e.g. plant cells or plant pollen), cells in seeds, or cells in tissues may be achieved. These outer layers could be native biological protections (e.g. complex exines for pollens), other types of cells, or general biological materials. In some cases, the needles are attached at an end on a surface. In some cases, pollen or seeds may have substantially thick and tough layers (intine and exine), which may render it more difficult to penetrate such materials, as compared with cell membranes, e.g., in mammalian cells.

### RELATED APPLICATIONS

This application claims the benefit of U.S. Provisional Patent Application Ser. No. 62/596,533, filed Dec. 8, 2017, entitled “Micro- and Nanoneedles for Plant Cell Penetration,” by Park, et al., incorporated herein by reference in its entirety.

### FIELD

The present invention generally relates to micro- and nanoneedles, e.g., for use in penetrating plant cells, or other cells.

### BACKGROUND

Substrate attached nanowires have been shown to effectively deliver biomolecules to in vitro cultured cells, where the cells are originally disassociated, suspended in solution, and allowed to fall to the substrate.

However, delivery of target biomolecules into cells with protective outer layers (e.g. plant cells), cells in seeds, or cells in tissues is challenging, for example, due to the need to protrude through additional layers of biological entities to reach the target location. These outer layers could be native biological protections (e.g. complex exines for pollens), other types of cells, or general biological materials. Accordingly, improvements are needed.

### SUMMARY

The present invention generally relates to micro- and nanoneedles, e.g., for use in penetrating plant cells, or other cells. The subject matter of the present invention involves, in some cases, interrelated products, alternative solutions to a particular problem, and/or a plurality of different uses of one or more systems and/or articles.

In one embodiment, the present invention is generally directed to an article, comprising an array of wires attached at a first end on a substrate, at least some of the wires comprising a first portion having a first diameter, and a second portion having a second diameter smaller than the first portion.

In another embodiment, the present invention is generally directed to an article, comprising a plant cell, one or more wires having a first end inserted into the plant cell, and a substrate that the one or more wires is attached to via a second end.

In yet another embodiment, the present invention is generally directed to a method, comprising inserting an array of wires into a plant cell, wherein at least some of the wires are attached to a surface at a first end and comprise a first portion having a first diameter, and a second portion having a second diameter smaller than the first portion.

In another aspect, the present invention encompasses methods of making one or more of the embodiments described herein, for example, a micro- or nanoneedle as discussed herein. In still another aspect, the present invention encompasses methods of using one or more of the embodiments described herein, for example, a micro- or nanoneedle as discussed herein.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates an array of needles inserted into targeted cells, in one embodiment of the invention;

FIGS. 2A-2D illustrates silicon needles, in another embodiment of the invention;

FIGS. 3A-3H illustrate needles made with single crystal quartz, in yet another embodiment of the invention;

FIGS. 4A-4E illustrate needles fabricated using photoresist, in still another embodiment of the invention;

FIGS. 5A-5D illustrate a method of making needles, in yet another embodiment of the invention;

FIGS. 6A-6E illustrate needles made with Si and amorphous SiO₂, in still another embodiment of the invention;

FIG. 7 illustrates delivery of biomolecules into cells, in one embodiment of the invention;

FIG. 8 illustrates electroporation or other electrically assisted delivery of biomolecules into cells, in another embodiment of the invention;

FIGS. 9A-9F illustrate needs with a metal exposed on the tip, in still another embodiment of the invention;

FIG. 10 illustrates localized electroporation delivery into cells, in yet another embodiment of the invention;

FIGS. 11A-11D illustrate a quartz nanoneedle array, in accordance with one embodiment of the invention;

FIGS. 12A-12D illustrate use of a quartz nanoneedle array, in accordance with another embodiment of the invention; and

FIGS. 13A-13D illustrate penetration of nanoneedles into plant pollen, in yet another embodiment of the invention.

### DETAILED DESCRIPTION

The present invention generally relates to micro- and nanoneedles, e.g., for use in penetrating plant cells, or other cells. In some embodiments, delivery of target biomolecules into cells with protective outer layers (e.g. plant cells or plant pollen), cells in seeds, or cells in tissues may be achieved. These outer layers could be native biological protections (e.g. complex exine for pollens), other types of cells, or general biological materials. In some cases, the needles are attached at an end on a surface. In some cases, pollen or seeds may have substantially thick and tough layers (e.g., intine and exine), which may render it more difficult to penetrate such materials, as compared with cell membranes, e.g., in mammalian cells.

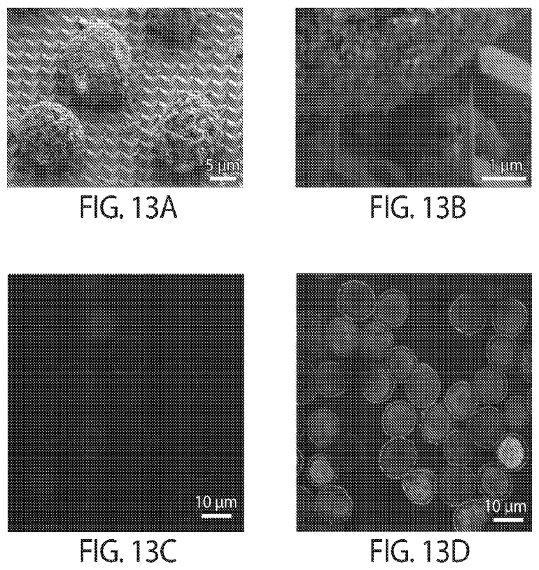

Certain embodiments are accordingly directed to systems and methods for penetrating protective outer layers of cells, especially in plants. For example, such cells may include seeds, spores, or pollen. Such cells typically will have stiff shapes, which may be sphere-like and/or may have spikes, spinules, gemmae, or other features. See, for example, FIG. 13A. In many cases, such features are distinctive enough that different species of plants can be identified or distinguished simply by examining the appearance of such features on pollen.

Unlike most mammalian cells, such cells may have multiple layers of protection, including the intine and the exine. The intine is mostly from materials such as pectin, cellulose, and/or hemicellulose, and is the inner protective layer. It can be 200 to 500 nm in thickness. The exine surrounds the intine, and is composed of stiff materials such as sporopollenin, a polymer of fatty acids, phenylpropanoids, phenolics, and carotenoids. It can be 500 to 800 nm in thickness. It is among the toughest of all known naturally-occurring biopolymers. In addition, there may be other surrounding layers of protection, such as cell walls and other cellulose structures outside of plant cells, or epidermis layers (e.g., 1, 2, or more layers of epidermis cells outside of plant tissues, e.g. seeds). These thus form protective outer layers surrounding the cytoplasmic portion of the cell. These can be 600-1200 nm thick, and is what allows pollen, spores, and the like to withstand environmental factors and propagate between plants. Accordingly, this outer layer thus is very touch and can be very difficult to penetrate. It does not deform easily and helps such cells retain their shape, even over geologic periods of time. Thus, certain embodiments of the invention are generally directed to systems and methods for penetrating such outer layers using nano- or microneedles.

In certain embodiments, such needles are formed from materials such as quartz (for example, single crystal quartz) that are tougher and less susceptible to deformation. Other materials are discussed in more details below. Single-crystal quartz, for instance, contains fewer (or no) grain boundaries that may present a weakness to the needles. Furthermore, the needles may be relatively long, e.g., at least 1 micrometer in length, so as to be able to penetrate the protective outer layers of cell (intine and exine).

Such needles, due to their length and material requirements, may be relatively thick or have relatively large diameters, e.g., to prevent breakage when applied against the outer layer. However, in some cases, the end of the needle may be smaller, e.g., to facilitate insertion. For instance, in one set of embodiments, the needle may comprise a first portion having a first, relatively constant diameter and a second portion, distal from the first portion, where the second portion is tapered, or has a second, relatively constant diameter smaller than the first diameter. As discussed below, the first portion may be relatively long (e.g., to prevent breakage) while the second portion may be relatively short (e.g., to facilitate insertion). In contrast, microneedles intended for piercing plasma membranes in mammalian cells would not typically need such dimensions in order to adequately pierce such membranes.

In addition, in one set of embodiments, additional force is applied to the needles to facilitate their penetration into cells such as seeds, spores, or pollen, in contrast to mammalian cells, which can often be pierced using only gravity or the weight of the cells. For instance, in certain cases, a force is applied to the cells and/or to the needles to cause penetration to occur. This force may be applied, for example, using centrifugal force, magnetically, electrically, mechanically, etc., for example, by applying pressure to the needles to cause them to penetrate. The pressure may be applied mechanically (e.g., using a press) or manually in some cases. The force applied may be at least 0.1 N, at least 0.2 N, at least 0.3 N, at least 0.5 N, at least 1 N, at least 2 N, at least 3 N, at least 5 N, at least 10 N, at least 20 N, at least 30 N, at least 40 N, at least 50 N, or at least 100 N, etc.

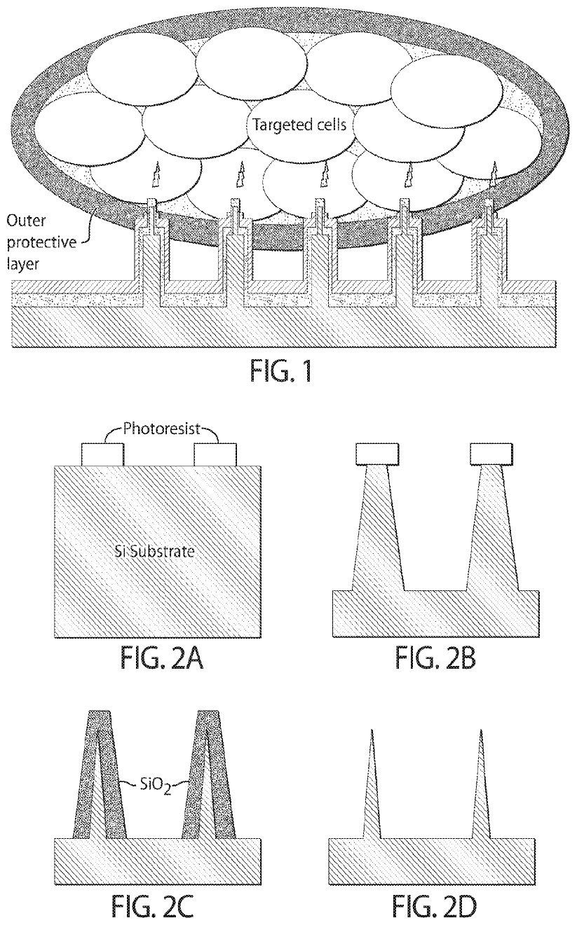

Some embodiments of the invention uses micro- and nanosized needles (also referred to herein as nanowires or NW) to penetrate through these additional layers to enable effective biomolecule delivery (FIG. 1, showing an example micro- or nanoneedle array for biomolecular delivery at tunable depths (figures herein are not to scale)). Electroporation can be used in some cases to increase the delivery efficiency through permeabilization of the membrane and/or can also increase the delivery selectivity by only metalizing the micro- or nanoneedle tips. By tuning the height and exposed metal area of the micro- or nanoneedles, biomolecules can be delivered at selective depths according to certain embodiments. The needles may be, for example, attached to a substrate or mounted to a manipulator; the latter may in some cases enhance interfacing with three-dimensional form factors or allow for uses in vivo. Certain embodiments are directed to applications, for example, such as genetic modification of pollen cells which have a greater than 1 micrometer cell wall and/or complex exine structures, genetic modification of plant seed stem cells wherein the cells are located greater than 10 micrometers from the seed surface, selective biomolecule delivery into particular layers of layered tissues such as the skin, regions of the brain (e.g. cortex) and plant leaf tissue, or the like.

In one set of embodiments, the nano- and micro-sized needles can be made of Si, amorphous SiO₂, single crystal quartz, polymers and hybrid materials, or the like. In some cases, photolithography, e-beam lithography, or other suitable techniques may be used to prepare the needles, including those described herein.

As a non-limiting example, for Si needles (FIG. 2), photolithography may first be performed on a Si wafer, e.g., to form circle structures with diameters ranging from 1 to 20 micrometers (FIG. 2A), then deep reactive ion etching (DRIE) or other techniques may be used to etch the Si into a pillar (in this particular example, ˜50 micrometers in height) (FIG. 2B). Etching parameters (e.g. pressure, gas flow, temperature, electrode bias power) can be used in some embodiments to taper the pillar into a needle during etching to achieve a smaller tip. However, in other embodiments, the pillar may be generally untampered. The needle diameter can be further reduced in certain cases by thermal oxidation of the surface Si to SiO₂ (FIG. 2C) and subsequent wet etching, e.g., using hydrofluoric acid (HF), to remove the oxide (FIG. 2D).

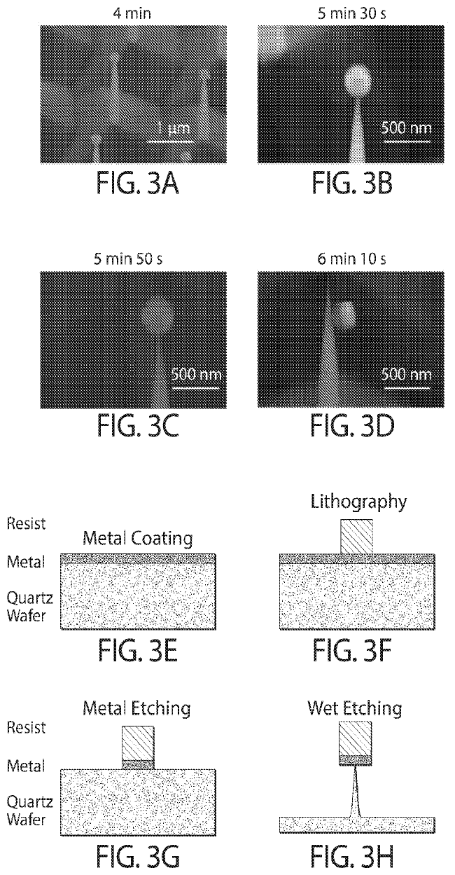

In another set of embodiments, the needles may be made with single crystal quartz via facet selective wet etching, as is shown in the example of FIG. 3. In some embodiments, for example, single crystal quartz can also be used to fabricate the microneedles (FIG. 3A-3D) via facet selective wet etching. The fabrication of quartz needles may start in this example from a single crystal Z-cut quartz wafer. First, a layer of metal (e.g., 60 nm of Cr) may be evaporated on the quartz wafer (FIG. 3E). Then, photolithography or e-beam lithography may be used to define arrays of circular features, e.g., with diameter ranging from 200 nm to 5 micrometers (FIG. 3F). The pitch between neighboring features can be constant or can vary, e.g., from 1 micrometer to 40 micrometers. In this example, the resist pattern may be transferred to metal dot arrays via dry or wet etching to selectively remove metal without resist on top (FIG. 3G). The metal dot arrays may be used as the hard mask for facet selective wet-etching of quartz using buffered hydrofluoric acid (FIG. 3H). After wet etching, the substrates may be rinsed in metal etchant to remove any remaining metal on the substrate and cleaned with piranha solution to remove contaminates. The needle height and wet-etching time may, in some cases, be linearly dependent on the diameter of metal hard mask and/or can also be tuned by selection of the etching solution.

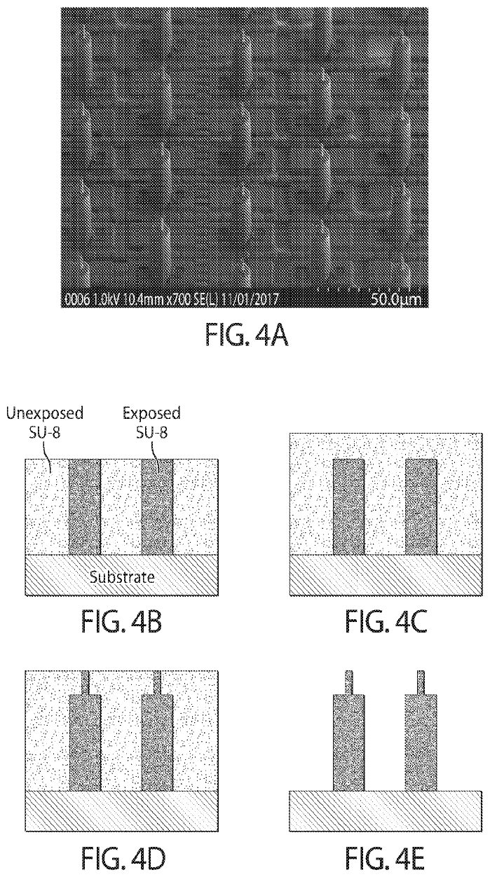

In yet another set of embodiments, the needles can also be made with SU-8 negative photoresist (FIG. 4A) through two lithographic steps. In one embodiment, a first layer of thick SU-8 (e.g., ˜50 micrometers) may be spun or otherwise deposited on the sample and exposed to form the base structure (FIG. 4B); then without developing, a second layer of thin SU-8 (e.g., 1˜10 micrometers) may be spun or otherwise deposited on the previous layer (FIG. 4C) and exposed to form a relatively small or sharp tip (FIG. 4D). Afterwards, both layers can be developed together, and/or may be hard-baked to create sharp tipped structures (FIG. 4E). Needles with variable heights can also be fabricated using different photoresist thicknesses in certain embodiments. FIG. 4 shows needles made with SU-8 via two-layer lithography

Thus, as discussed herein, certain embodiments are generally directed to micro- or nanoneedles attached on one end to a surface, where the needles include a first portion having a first, relatively constant diameter and a second portion, e.g., distal from the first portion, having a second, relatively constant diameter smaller than the first diameter. In some cases, the first portion may comprise at least 50%, at least 60%, at least 70%, at least 80%, at least 90%, or at least 95% of the length of the needle (or wire). In addition, in some cases, the first portion may comprise no more than 95%, no more than 90%, no more than 80%, no more than 70%, no more than 60%, or no more than 50% of the length of the needle (or wire). Combinations of any of these are also possible.

In some cases, the second portion has a diameter that is less than 50%, less than 40%, less than 30%, less than 20%, less than 10%, or less than 5% of the diameter of the first portion. In some cases, the second portion has a diameter that is no more than 5%, no more than 10%, no more than 15%, no more than 20%, no more than 30%, no more than 40%, or no more than 50% of the diameter of the first portion. Combinations of any of these are also possible in certain embodiments.

In some cases, the first portion and/or the second portion may each independently have an average length of at least about 0.1 micrometers, at least about 0.2 micrometers, at least about 0.3 micrometers, at least about 0.5 micrometers, at least about 0.7 micrometers, at least about 1 micrometer, at least about 2 micrometers, at least about 3 micrometers, at least about 5 micrometers, at least about 7 micrometers, at least about 10 micrometers, at least about 20 micrometers, at least about 30 micrometers, at least about 50 micrometers, or at least about 100 micrometers. In some cases, the first portion and/or the second portion may have an average length of no more than about 100 micrometers, no more than about 70 micrometers, no more than about 50 micrometers, no more than about 30 micrometers, no more than about 20 micrometers, no more than about 10 micrometers, no more than about 7 micrometers, no more than about 5 micrometers, no more than about 3 micrometers, no more than about 2 micrometers, no more than about 1 micrometer, no more than about 0.7 micrometers, no more than about 0.5 micrometers, no more than about 0.3 micrometers, no more than about 0.2 micrometers, or no more than about 0.1 micrometers. Combinations of any of these are also possible in some embodiments.

The first portion and/or the second portion may each independently have any suitable diameter, or narrowest dimension if not circular. In some cases, the portions may each independently have an average diameter of at least about 10 nm, at least about 30 nm, at least about 50 nm, at least about 70 nm, at least about 100 nm, at least about 200 nm, at least about 300 nm, at least about 500 nm, at least about 1 micrometer, at least about 2 micrometers, at least about 3 micrometers, at least about 5 micrometers, at least about 10 micrometers, etc., and/or the portions may have an average diameter of no more than about 10 micrometers, no more than about 7 micrometers, no more than about 5 micrometers, no more than about 3 micrometers, no more than about 2 micrometers, no more than about 1 micrometers, no more than about 500 nm, no more than about 300 nm, no more than about 200 nm, no more than about 100 nm, no more than about 70 nm, no more than about 50 nm, no more than about 30 nm, no more than about 20 nm, or no more than about 10 nm, or any combination of these.

If more than one needle is present, e.g. attached on one end to a surface, the needles may each independently have the same or different dimensions, e.g., as discussed herein. Non-limiting examples of needle dimensions are discussed herein.

In addition, in some cases, there may be 3 or more such portions on the needle or wire, each having dimensions such as those discussed herein. For example, a micro- or nanoneedle may have a first portion having a first diameter, a second portion having a second diameter smaller than the first diameter (e.g., having the above percentages), and a third portion having a third diameter smaller than the second diameter (e.g., having the above percentages), etc.

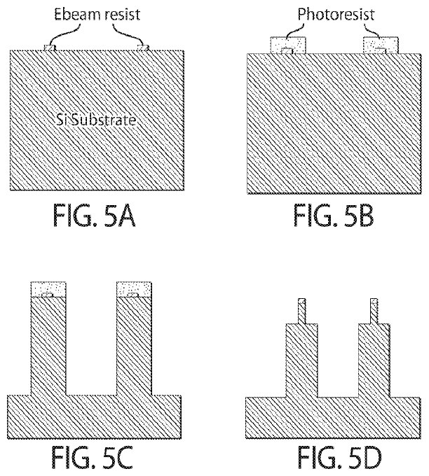

Another non-limiting example of making such needles is now described with respect to FIG. 5, showing needles made with Si via two-layer lithography and DRIE. In this example, a two-layer lithography can also be performed on Si with DRIE (FIG. 5): a negative ebeam resist (e.g. HSQ) may be spun on the Si substrate and exposed into nanoscale circles (FIG. 5A), which can be used as a later etching mask. A photoresist may then be spun onto the Si substrate and patterned into microscale circles (FIG. 5B). The sample can then be dry etched in the DRIE machine to form the base structure (e.g., ˜50 micrometers in height) using the photoresist as the etch mask (FIG. 5C). The photoresist may then be removed in solvent and in some cases, the sample may be etched again with the ebeam resist as etching mask, e.g., to form sharp tips (FIG. 5D).

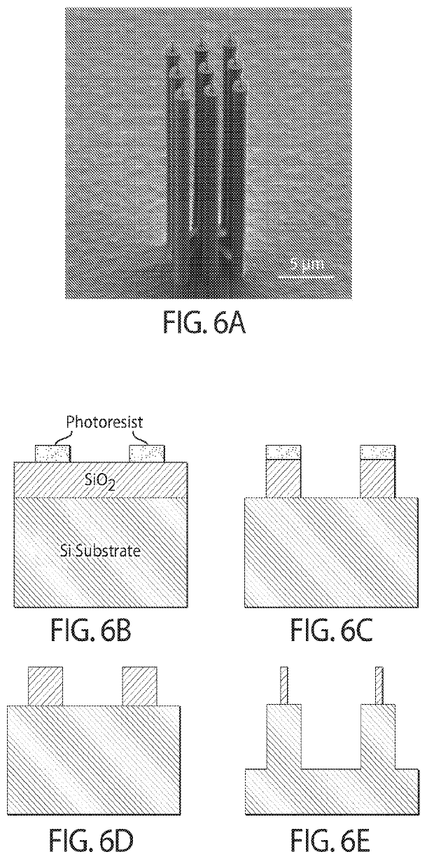

In another set of embodiments, the needles can also be made with Si and amorphous SiO₂ (FIG. 6A) via mixed dry etching and wet etching. In some cases, a layer of amorphous SiO₂ (e.g., 1˜10 micrometers) may be first deposited onto the Si sample via chemical vapor deposition (CVD), then photolithography may be performed to produce micro circles as etching masks (FIG. 6B). Using the photoresist as etch mask, the oxide may be dry etched into cylinders (FIG. 6C), then using the oxide cylinders as etch masks the Si substrate may be etched into base structures (˜50 micrometers in height) (FIG. 6D). Afterwards, wet etchant (e.g. hydrofluoric acid, HF) may be used to etch the SiO₂ cylinder into sharp tips without affecting the Si base structure (FIG. 6E). FIG. 6 illustrates needles made with Si and SiO₂ via mixed dry and wet etching.

The substrate may be formed of the same or different materials as the wires. For example, the substrate may comprise silicon, silicon oxide, silicon nitride, silicon carbide, iron oxide, aluminum oxide, iridium oxide, tungsten, stainless steel, silver, platinum, gold, gallium, germanium, or any other materials described herein that a wire may be formed from. In one embodiment, the substrate is formed from a semiconductor.

In some embodiments, arrays of wires on a substrate may be obtained by growing wires from a precursor material. The wires may be, for example, nanowires, nanoneedles, microneedles, etc., e.g., as described herein. As a non-limiting example, chemical vapor deposition (CVD) may be used to grow wires by placing or patterning catalyst or seed particles (typically with a diameter of 1 nm to a few hundred nm) atop a substrate and adding a precursor to the catalyst or seed particles. When the particles become saturated with the precursor, wires can begin to grow in a shape that minimizes the system's energy. By varying the precursor, substrate, catalyst/seed particles (e.g., size, density, and deposition method on the substrate), and growth conditions, wires can be made in a variety of materials, sizes, and shapes, at sites of choice.

In certain embodiments, arrays of wires on a substrate may be obtained by growing wires using a top-down process that involves removing predefined structures from a supporting substrate. As a non-limiting example, the sites where wires are to be formed may be patterned into a soft mask and subsequently etched to develop the patterned sites into three-dimensional wires. Methods for patterning the soft mask include, but are not limited to, photolithography and electron beam lithography. The etching step may be either wet or dry.

Some aspects of the invention are directed to coating the substrate with a positive resist. As used herein, “positive resist” refers to a material that becomes soluble to a resist developer after being exposed to a beam of photons or electrons. When a beam of photons is used, the technique is generally termed photolithography, and when a beam of electrons is used, the technique is generally referred to as electron beam lithography. Examples of positive resists used in photolithography include, but are not limited to, poly(methyl methacrylate) (PMMA) and SPR220, S1800, and ma-P1200 series photoresists. Other examples of photoresists include, but are not limited to, SU-8, S1805, LOR 3A, poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, Shipley 1400-37, or the like. Examples of positive resists used in electron beam lithography include, but are not limited to, PMMA, ZEP 520, APEX-E, EBR-9, and UV5. In some embodiments, portions of the resist may be exposed to light (visible, UV, etc.), electrons, ions, X-rays, etc. (e.g., projected onto the photoresist), and the exposed portions can be etched away (e.g., using suitable etchants, plasma, etc.) to produce a suitable pattern.

Certain aspects of the invention are directed to methods comprising coating the substrate with a negative resist. As used herein, “negative resist” refers to a material that becomes less soluble to a resist developer after being exposed to a beam of photons or electrons. Several non-limiting examples of negative resists used in photolithography include SU-8 series photoresists, KMPR 1000, and UVN30. Additional non-limiting examples of negative resists used in electron beam lithography include hydrogen silsesquioxane (HSQ) and NEB-31.

It should be appreciated that any positive resist, negative resist, or resist developer known in the art may be used. Resist developers for photolithography include aqueous solutions with either an organic compound such as tetramethylammonium hydroxide or an inorganic salt such as potassium hydroxide, and they may also contain surfactants. Resist developers for electron beam lithography may include methyl isobutyl ketone and isopropyl alcohol.

In addition, certain aspects of the invention relate to methods that comprise exposing a substrate that has been coated with a positive or negative resist to a pre-determined pattern of photon or electron beams to form a pattern of nanosites. In some aspects of the invention, the pattern may be a repeating pattern. In certain cases, the pattern may be a rectangular pattern.

For example, the wires may be regularly positioned within a rectangular grid with periodic spacing, e.g., having a periodic spacing of at least about 0.01 micrometers, at least about 0.03 micrometers, at least about 0.05 micrometers, at least about 0.1 micrometers, at least about 0.3 micrometers, at least about 0.5 micrometers, at least about 1 micrometer, at least about 2 micrometers, at least about 3 micrometers, at least about 5 micrometers, at least about 10 micrometers, etc. In some cases, the periodic spacing may be no more than about 10 micrometers, no more than about 5 micrometers, no more than about 3 micrometers, no more than about 1 micrometer, no more than about 0.5 micrometers, no more than about 0.3 micrometers, no more than about 0.1 micrometers, no more than about 0.05 micrometers, no more than about 0.03 micrometers, no more than about 0.01 micrometers, etc. Combinations of these are also possible, e.g., the array may have a periodic spacing of wires of between about 0.01 micrometers and about 0.03 micrometers.

In some cases, the wires (whether regularly or irregularly spaced) may be positioned on the substrate such that the average distance between a wire and its nearest neighboring wire is at least about 0.01 micrometers, at least about 0.03 micrometers, at least about 0.05 micrometers, at least about 0.1 micrometers, at least about 0.3 micrometers, at least about 0.5 micrometers, at least about 1 micrometer, at least about 2 micrometers, at least about 3 micrometers, at least about 5 micrometers, at least about 10 micrometers, etc. In some cases, the distance may be no more than about 10 micrometers, no more than about 5 micrometers, no more than about 3 micrometers, no more than about 1 micrometer, no more than about 0.5 micrometers, no more than about 0.3 micrometers, no more than about 0.1 micrometers, no more than about 0.05 micrometers, no more than about 0.03 micrometers, no more than about 0.01 micrometers, etc. In some cases, the average distance may fall within any of these values, e.g., between about 0.5 micrometers and about 2 micrometers.

Some embodiments of the invention are directed to developing a positive resist so that the pattern of nanosites is converted to a pattern of nanoholes in the resist. In some of those embodiments, a hard etch mask is subsequently deposited into each of the nanoholes. In some embodiments, the hard etch mask comprises or consists essentially of a metal such as aluminum oxide, aluminum, or a combination thereof. The resist is then uplifted or removed, leaving a pattern of nanospots each covered by a hard etch mask, the pattern of the nanospots being the same as the pattern of the nanoholes. In certain embodiments, the substrate is then etched to a desired depth to yield an array of wires, the hard etch masks protecting the covered nanospots and substrate lying beneath from being etched. Wires are thereby formed at a plurality of positions arranged in two dimensions and have a local density of 0.001 to 10 wires per micrometer² (μm²). FIG. 2 illustrates large scale fabrication of NW arrays using an embodiment of the invention.

Some embodiments of the invention are directed to developing a negative resist so that the pattern of nanosites is converted to a pattern of masked nanospots. In some cases, the substrate is subsequently etched to a desired depth to yield an array of wires at the masked nanospots, thereby forming wires at a plurality of positions arranged in two dimensions and having a local density of 0.001-10 wires per micrometer² (μm²), or other densities as described in detail herein.

Certain embodiments of the invention are generally directed to biomolecule delivery. A variety of molecules may be delivered in various embodiments of the invention, including proteins, nucleic acids, hormones, peptides, DNA, RNA, antibodies, enzymes, or the like.

In some embodiments, the needles can be used to mechanically penetrate through additional biological material layers for direct delivery of biomolecules and/or they can be coated with metal for electrically-assisted delivery, e.g., using electroporation, electrophoresis, direct current, or other techniques.

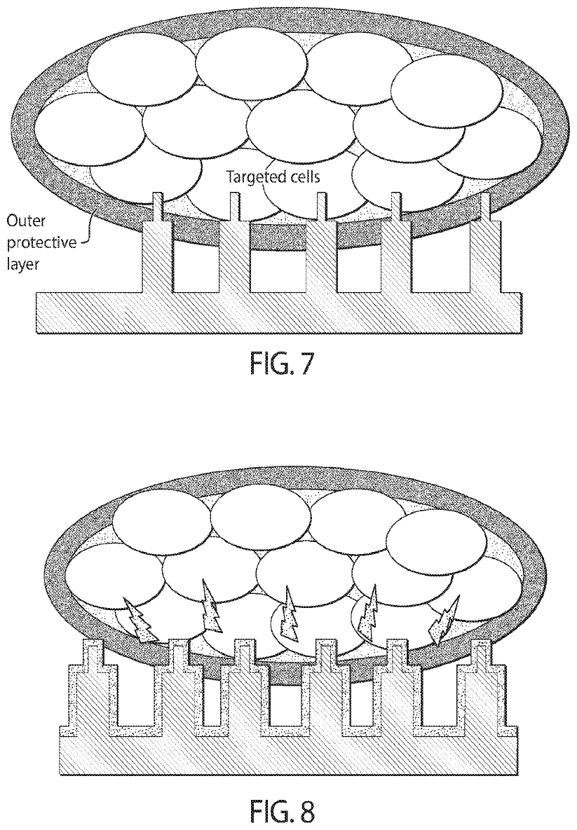

In one set of embodiments, as is shown in FIG. 7, direct penetration delivery of biomolecules may be used. In some embodiments, for direct delivery (FIG. 7), the needles can be brought into the vicinity of the target cell (e.g., a plant seed with internal target stem cells) with micromanipulators, mechanical pushing, centrifugation, etc., to penetrate the outer protective layer(s) of biological materials (e.g. the outside cells of the seed). In some cases, the cells may be placed or deposited onto the needles. In some cases, a force may be applied to the cells and/or to the needles, e.g., to cause the needles to enter the cells.

The biomolecules to be delivered can be coated directly on the needles and/or can be suspended in solution. Upon penetration, the biomolecules may be released from the surface or influx into the hole created via penetration, e.g., for delivery into the target cells.

In another set of embodiments, e.g., as is shown in FIG. 8, electroporation or other electrically assisted delivery of biomolecules may be used. For example, for electroporation assisted delivery (FIG. 8), the needles may be first coated with a thin layer of metal (e.g. Pt or Au) via sputtering, evaporation, atomic layer deposition (ALD), or the like, e.g., to make them conductive microelectrodes. They may be brought into mechanical contact with a target cell, e.g., as discussed above with respect to direct penetration. An electrical signal may be applied to the needles, e.g., to generate holes (electroporation) in the target cell membrane for delivery. In some cases, the biomolecules to be delivered can be coated directly on the needles and/or can be suspended in solution. Upon electroporation, the biomolecules may be released from the surface or influx into the holes, or otherwise delivered into the target cells.

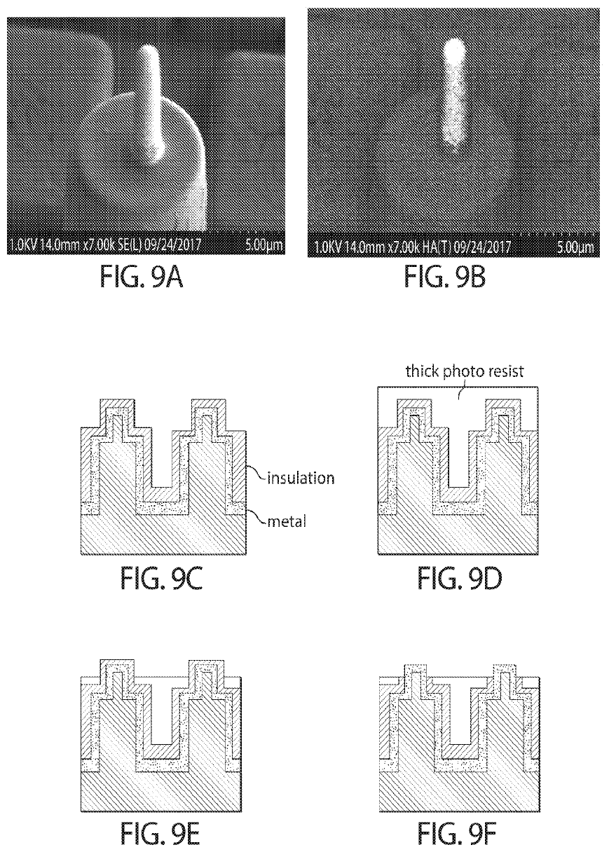

In one set of embodiments, localized electroporation rather than whole area electroporation may be used. FIG. 9 shows fabrication of tall microelectrodes with metal exposed only on the tip. In some cases, the needles can be modified so that they only have a conductive tip exposed (FIGS. 9A and 9B). First a conformal metal layer may be deposited on the needle with tip structures via sputtering, evaporation or ALD. An insulation layer (e.g. SiO₂, Si₃N₄, HfO₂, etc.) may then be deposited around the structure via sputtering, evaporation, ALD, or other similar techniques (FIG. 9C). A photoresist (e.g. SU-8) can then be spun on the sample to cover all the structures (FIG. 9D), and O₂ plasma etching or chemical developing, or other techniques, may be used to remove a layer on the top to expose part of the sharp tip only (FIG. 9E). Wet etching or other etching techniques may then be used to remove the insulation layer only at the exposed part of the sharp tip (FIG. 9F). The structure may then be ready for use after resist removal. This process can also be applied to any of the geometries previously described, in other embodiments.

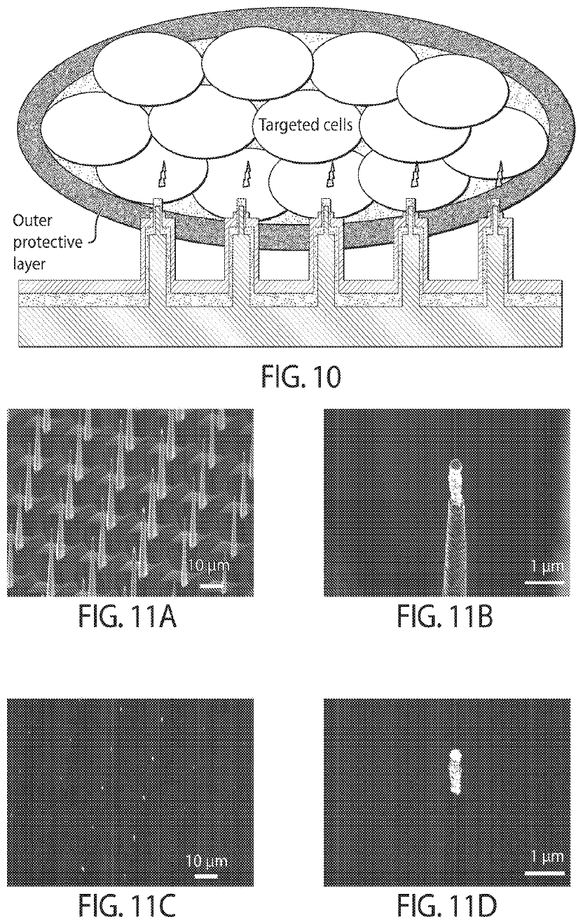

In one set of embodiments, localized electroporation delivery may be used. As is shown in FIG. 10, needles may be brought into mechanical contact with the target cell, e.g., as discussed herein. An electrical signal may be then applied to the needle microelectrode. Since metal is exposed only at the tip of the needle, only the cells in close proximity to the tip may be electroporated (or other electrically-based techniques such as those discussed herein) and biomolecules can be selectively delivered. Similarly, the desired target biomolecule can be coated directly on the needles or can be suspended in solution. FIG. 10 shows localized electroporation assisted delivery of biomolecules.

In one embodiment, both wires and the substrate are electrically conductive. The wires may be distributed on the substrate, e.g., as discussed herein. In some embodiments, the wires can be coated with metals, which may enhance their electric conductivity. Molecular delivery can be achieved, for example, by applying a current or voltage waveform between the substrate and an electrode in the bath solution. Almost all of the cells atop the substrate are electroporated and remain viable. The amplitude required for biomoleular delivery may be only a few volts. For example, the amplitude may be at least 0.1 V, at least 0.2 V, at least 0.3 V, at least 0.5 V, at least 1 V, at least 2 V, at least 3 V, at least 5 V, or at least 10 V, and/or the amplitude may be less than 10 V, less than 5 V, less than 3 V, less than 2 V, less than 1 V, less than 0.5 V, less than 0.3 V, less than 0.2 V, or less than 0.1 V. The insulating layer coated over the wires, in some cases, may be formed of a material with low cytoxicity (e.g., silicon oxide, aluminum oxide, and silicon nitride).

In another embodiment, the wires are present on a substrate as individual sets, e.g., to allow site-specific delivery of biomolecules into cells. Each individual set may in some cases be electrically insulated from other sets. Wires in the same set may be electrically connected and addressable independently from other sets. In this way, cell-cell or cell-network interactions can be studied by providing specific perturbations to individual cells within an interacting system.

The substrate may comprise one or more upstanding wires, which may include nanowires, nanoneedles, microneedles, etc. On average, the upstanding wires may form an angle with respect to a substrate of between about 80° and about 100°, between about 85° and about 95°, or between about 88° and about 92°. In some cases, the average angle is about 90°. As used herein, the term “nanowire” (or “NW”) refers to a material in the shape of a wire or rod having a diameter in the range of 1 nm to 1 micrometer (μm). The wires may be formed from materials with low cytotoxicity; suitable materials include, but are not limited to, silicon, silicon oxide, silicon nitride, silicon carbide, iron oxide, aluminum oxide, iridium oxide, tungsten, stainless steel, silver, platinum, and gold. Other suitable materials include aluminum, copper, molybdenum, tantalum, titanium, nickel, tungsten, chromium, or palladium. In some embodiments, the wires comprises or consists essentially of a semiconductor. Typically, a semiconductor is an element having semiconductive or semi-metallic properties (i.e., between metallic and non-metallic properties). An example of a semiconductor is silicon. Other non-limiting examples include elemental semiconductors, such as gallium, germanium, diamond (carbon), tin, selenium, tellurium, boron, or phosphorous. In other embodiments, more than one element may be present in the wires as the semiconductor, for example, gallium arsenide, gallium nitride, indium phosphide, cadmium selenide, etc.

The size and density of the wires in the wire arrays may be varied; the lengths, diameters, and density of the wires can be configured to permit adhesion and penetration of cells. In some embodiments, the length of the wires can be 0.1-10 micrometers (μm). In some cases, the diameter of the wires can be 50-300 nm. In certain embodiments, the density of the wires can be 0.05-5 NWs per micrometer (μm²). Other examples are discussed below.

The wires may have any suitable length, as measured moving away from the substrate. The wires may have substantially the same lengths, or different lengths in some cases. For example, the wires may have an average length of at least about 0.1 micrometers, at least about 0.2 micrometers, at least about 0.3 micrometers, at least about 0.5 micrometers, at least about 0.7 micrometers, at least about 1 micrometer, at least about 2 micrometers, at least about 3 micrometers, at least about 5 micrometers, at least about 7 micrometers, at least about 10 micrometers, at least about 20 micrometers, at least about 30 micrometers, at least about 50 micrometers, or at least about 100 micrometers. In some cases, the wires may have an average length of no more than about 100 micrometers, no more than about 70 micrometers, no more than about 50 micrometers, no more than about 30 micrometers, no more than about 20 micrometers, no more than about 10 micrometers, no more than about 7 micrometers, no more than about 5 micrometers, no more than about 3 micrometers, no more than about 2 micrometers, no more than about 1 micrometer, no more than about 0.7 micrometers, no more than about 0.5 micrometers, no more than about 0.3 micrometers, no more than about 0.2 micrometers, or no more than about 0.1 micrometers. Combinations of any of these are also possible in some embodiments.

The wires may also have any suitable diameter, or narrowest dimension if the wires are not circular. The wires may have substantially the same diameters, or in some cases, the wires may have different diameters. In some cases, the wires may have an average diameter of at least about 10 nm, at least about 30 nm, at least about 50 nm, at least about 70 nm, at least about 100 nm, at least about 200 nm, at least about 300 nm, at least about 500 nm, at least about 1 micrometer, at least about 2 micrometers, at least about 3 micrometers, at least about 5 micrometers, at least about 10 micrometers, etc., and/or the wires may have an average diameter of no more than about 10 micrometers, no more than about 7 micrometers, no more than about 5 micrometers, no more than about 3 micrometers, no more than about 2 micrometers, no more than about 1 micrometers, no more than about 500 nm, no more than about 300 nm, no more than about 200 nm, no more than about 100 nm, no more than about 70 nm, no more than about 50 nm, no more than about 30 nm, no more than about 20 nm, or no more than about 10 nm, or any combination of these.

In addition, in some cases, the density of wires on the substrate, or on a region of the substrate defined by wires, may be at least about 0.01 wires per square micrometer, at least about 0.02 wires per square micrometer, at least about 0.03 wires per square micrometer, at least about 0.05 wires per square micrometer, at least about 0.07 wires per square micrometer, at least about 0.1 wires per square micrometer, at least about 0.2 wires per square micrometer, at least about 0.3 wires per square micrometer, at least about 0.5 wires per square micrometer, at least about 0.7 wires per square micrometer, at least about 1 wire per square micrometer, at least about 2 wires per square micrometer, at least about 3 wires per square micrometer, at least about 4 wires per square micrometer, at least about 5 wires per square micrometer, etc. In addition, in some embodiments, the density of wires on the substrate may be no more than about 10 wires per square micrometer, no more than about 5 wires per square micrometer, no more than about 4 wires per square micrometer, no more than about 3 wires per square micrometer, no more than about 2 wires per square micrometer, no more than about 1 wire per square micrometer, no more than about 0.7 wires per square micrometer, no more than about 0.5 wires per square micrometer, no more than about 0.3 wires per square micrometer, no more than about 0.2 wires per square micrometer, no more than about 0.1 wires per square micrometer, no more than about 0.07 wires per square micrometer, no more than about 0.05 wires per square micrometer, no more than about 0.03 wires per square micrometer, no more than about 0.02 wires per square micrometer, or no more than about 0.01 wires per square micrometer.

The wires may be regularly or irregularly spaced on the substrate. For example, the wires may be positioned within a rectangular grid with periodic spacing, e.g., having a periodic spacing of at least about 0.01 micrometers, at least about 0.03 micrometers, at least about 0.05 micrometers, at least about 0.1 micrometers, at least about 0.3 micrometers, at least about 0.5 micrometers, at least about 1 micrometer, at least about 2 micrometers, at least about 3 micrometers, at least about 5 micrometers, at least about 10 micrometers, etc. In some cases, the periodic spacing may be no more than about 10 micrometers, no more than about 5 micrometers, no more than about 3 micrometers, no more than about 1 micrometer, no more than about 0.5 micrometers, no more than about 0.3 micrometers, no more than about 0.1 micrometers, no more than about 0.05 micrometers, no more than about 0.03 micrometers, no more than about 0.01 micrometers, etc. Combinations of these are also possible, e.g., the array may have a periodic spacing of wires of between about 0.01 micrometers and about 0.03 micrometers.

In some cases, the wires (whether regularly or irregularly spaced) may be positioned on the substrate such that the average distance between a wire and its nearest neighboring wire is at least about 0.01 micrometers, at least about 0.03 micrometers, at least about 0.05 micrometers, at least about 0.1 micrometers, at least about 0.3 micrometers, at least about 0.5 micrometers, at least about 1 micrometer, at least about 2 micrometers, at least about 3 micrometers, at least about 5 micrometers, at least about 10 micrometers, etc. In some cases, the distance may be no more than about 10 micrometers, no more than about 5 micrometers, no more than about 3 micrometers, no more than about 1 micrometer, no more than about 0.5 micrometers, no more than about 0.3 micrometers, no more than about 0.1 micrometers, no more than about 0.05 micrometers, no more than about 0.03 micrometers, no more than about 0.01 micrometers, etc. In some cases, the average distance may fall within any of these values, e.g., between about 0.5 micrometers and about 2 micrometers.

In one aspect, the electrical device includes a substrate having a surface, which is coated with an electrically insulted layer, and a plurality of electrically conductive wires, each of which, having a first end and a second end, is coated with an electrically insulating layer except for the first and second ends, the first end being attached to the surface and the second end being coated with an electrically conductive layer. Each wire can be individually addressable, for example, by a voltage waveform. The wires may include, for example, nanowires, nanoneedles, microneedles, etc., such as those discussed herein.

The wires used herein can also be made, in some embodiments, of one or more of a semiconductor (e.g., Si and Ge), a compound semiconductor, a metal oxide (e.g., ZnO), a metal (e.g., Au, Ag, Ir, Pt), carbon, boron nitride, or a combination thereof.

The term “compound semiconductor” refers to a semiconductive compound formed of two or more elements such as IV-IV semiconductors (e.g., SiC and SiGe), III-V semiconductors (e.g., AlN, AlP, AlGaAs, GaN, GaAs, InP, and InGaAs), II-V semiconductors (e.g., Zn₃Sb₂ and Cd₃As₂), II-VI semiconductors (e.g., CdS, CdSe, CdTe, IV-VI semiconductors (e.g., SnS and PbSnTe), I-VI semiconductors (e.g., Cu₂S), I-VII semiconductors (e.g., CuCl), and oxide semiconductors (e.g., SnO₂, CuO, and Cu₂O). Unless stated otherwise, the semiconductor used for the electrical device may be in its intrinsic form (i.e., pure form) and/or in a doped form (i.e., containing one or more dopants). Such combinations may include a mixture, an alloy, or a suitable reaction product of two or more components.

The electrically insulating layer, e.g., on a wire, may be formed in some embodiments of an inorganic material such as an oxide (e.g., silica, alumina, and hafnium oxide) or a nitride (e.g., silicon nitride). In some cases, the electrically insulating layer may be formed of an organic material such as Parylene (e.g., Parylene C, N, AF-4, SF, HT, A, AM, VT-4, or CF), polydimethylsiloxane, methyl methacrylate, a photoresist (e.g., SU-8), an electron beam resist (e.g., polymethylmethacrylate, ZEP-520, and hydrogen silsesquioxane), or the like, including combinations thereof.

For example, the electrically conductive layer may be formed of a semiconductor (e.g., Si and Ge), a compound semiconductor, a metal (e.g., Ag, Au, Pt, Ni, Al, Pd, W, Ti, and Cr), a metal oxide (e.g., indium tin oxide), a metal nitride (e.g., titanium nitride), or a combination thereof (e.g., a metal silicide).

In some embodiments, the electrically conductive layer can include a metal top layer and a metal silicide intermediate layer between the metal top layer and the second end. The metal silicide can be a silicide of Pt, Ni, W, Pd, Ti, Cr, Yb, Er, Tb, Dy, Gd, Ho, Y, Hf, Zr, Ta, Co, V, Mo, Rh, Ir, or a combination thereof. Examples of silicide include but are not limited to PtSi, Pt₂Si, NiSi, Ni₂Si, NiSi₂, WSi₂, Pd₂Si, TiSi₂, CrSi₂, YbSi₂, ErSi₂, TbSi₂, DySi₂, GdSi₂, HoSi₂, YSi₂, HfSi, ZrSi₂, TaSi₂, CoSi₂, VSi₂, CoSi, MoSi₂, RhSi, Ir₂Si₃, IrSi, and IrSi₃.

The plurality of electrically conductive wires in a device can include in some embodiments a first plurality of electrically conductive wires and a second plurality of electrically conductive wires. The first plurality of wires can be in electrical communication with each other. They can each be electrically insulated from the second plurality of electrically conductive wires. The first and second pluralities of wires may be the same or different in terms of composition and configuration. Alternatively, at least two of the plurality of wires can be electrically insulated from each other.

The substrate can be formed of a semiconductor (e.g., Si), a compound semiconductor (e.g., GaAs, InP, GaN, and GaP), or diamond. The substrate may be formed of the same, or different materials, than the wire.

In certain aspects, the substrate may comprise more than one region of wires, e.g., patterned as discussed herein. For example, a pre-determined pattern of photons or electrons may be used to produce a substrate comprising a first region of wires and a second region of wires. In addition, in some cases, more than two such regions of wires may be produced on a substrate. For example, there may be at least 3, at least 6, at least 10, at least 15, at least 20, at least 50, or at least 100 separate regions of wires on a substrate. In some cases, the regions are separate from each other. Any number of wires may be present in a region, e.g., at least about 10, at least about 20, at least about 50, at least about 100, at least about 300, at least about 1000, etc. The wires may be present in any suitable configuration or array, e.g., in a rectangular or a square array.

The wires in a first region and a second region may be the same, or there may be one or more different characteristics between the wires. For example, the wires in the first region and the second region may have different average diameters, lengths, densities, biological effectors, or the like. If more than two regions of wires are present on the substrate, each of the regions may independently be the same or different.

In some embodiments, arrays of wires on a substrate may be obtained by growing wires from a precursor material. As a non-limiting example, chemical vapor deposition (CVD) may be used to grow wires by placing or patterning catalyst or seed particles (typically with a diameter of 1 nm to a few hundred nm) atop a substrate and adding a precursor to the catalyst or seed particles. When the particles become saturated with the precursor, wires can begin to grow in a shape that minimizes the system's energy. By varying the precursor, substrate, catalyst/seed particles (e.g., size, density, and deposition method on the substrate), and growth conditions, wires can be made in a variety of materials, sizes, and shapes, at sites of choice.

In certain embodiments, arrays of wires on a substrate may be obtained by growing wires using a top-down process that involves removing predefined structures from a supporting substrate. As a non-limiting example, the sites where wires are to be formed may be patterned into a soft mask and subsequently etched to develop the patterned sites into three-dimensional wires. Methods for patterning the soft mask include, but are not limited to, photolithography and electron beam lithography. The etching step may be either wet or dry.

As mentioned, wires can be used to deliver molecules to any eukaryotic or prokaryotic cell, in certain aspects of the invention. The wires may include, for example, nanowires, nanoneedles, microneedles, etc., such as those discussed herein. The system may include a first molecule to be delivered (e.g., a small molecule, a nucleic acid, a protein, or a polysaccharide), either attached to the linker permanently (i.e., not detachable from the linker in a cell) or reversibly (i.e., detachable from the linker in a cell). The system may further include a second molecule to be delivered (e.g., a small molecule, a nucleic acid, a protein, or a polysaccharide), either attached to the linker permanently or reversibly. The first and second molecules are different molecules. They may be connected to the same wire or different wires. wires on the same substrate may have different silane linkers for delivering different molecules. The wires attached to the surface may extend along a uniform direction, such as a substantially vertical direction (i.e., 60-90 degrees) to the substrate surface. The wires can have any of the lengths or materials described herein. In some cases, for example, the silicon-containing material in the surface layer of each wire can be elemental silicon, silicon oxide, silicon nitride, or silicon carbide. The silane linker can be propylaminosilane, for example.

Certain embodiments relate to a method of delivering an exogenous molecule into a cell. This may include providing a substrate having a surface and a plurality of wires (e.g., as discussed herein) adhered to the surface, in which at least some of the wires has a covalently bound linker to which a molecule to be delivered is attached, and contacting the nanowires with a cell to allow penetration of the nanowires into the cell, whereby the molecule is delivered into the cell. The systems described herein can be used for delivering various molecules both in vitro and in vivo.

In some cases, exogenous molecules (e.g., RNAs, peptides, and proteins) can be delivered into cells with unexpectedly high efficiency. Also, given the high delivery efficiency, massive parallel screening (i.e., assaying different molecules in the same cell or different cells in a short period of time) can be achieved. For example, one can use the delivery system in proteomics by attaching one type of protein antibodies to wires contained in one location of the substrate and then screening for proteins in a specific cell type.

In one set of embodiments, at least some of the wires may be used to deliver a molecule of interest into a cell, e.g., through insertion of a wire into the cell. In certain embodiments of the invention, at least some of the wires may undergo surface modification so that molecules of interest can be attached to them. It should be appreciated that the wires can be complexed with various molecules according to any method known in the art. It should also be appreciated that the molecules connected to different wires may be distinct. In some embodiments, a wire may be attached to a molecule of interest through a linker. The interaction between the linker and the wire may be covalent, electrostatic, photosensitive, or hydrolysable. As a specific non-limiting example, a silane compound may be applied to a wire with a surface layer of silicon oxide, resulting in a covalent Si—O bond. As another specific non-limiting example, a thiol compound may be applied to a wire with a surface layer of gold, resulting in a covalent Au—S bond. Examples of compounds for surface modification include, but are not limited to, aminosilanes such as (3-aminopropyl)-trimethoxysilane, (3-aminopropyl)-triethoxysilane, 3-(2-aminoethylamino)propyl-dimethoxymethylsilane, (3-aminopropyl)-diethoxy-methylsilane, [3-(2-aminoethylamino)propyl]trimethoxysilane, bis[3-(trimethoxysilyl)propyl]amine, and (11-aminoundecyl)-triethoxysilane; glycidoxysilanes such as 3-glycidoxypropyldimethylethoxysilane and 3-glycidyloxypropyl)trimethoxysilane; mercaptosilanes such as (3-mercaptopropyl)-trimethoxysilane and (11-mercaptoundecyl)-trimethoxysilane; and other silanes such as trimethoxy(octyl)silane, trichloro(propyl)silane, trimethoxyphenylsilane, trimethoxy(2-phenylethyl)silane, allyltriethoxysilane, allyltrimethoxysilane, 3-[bis(2-hydroxyethyl)amino]propyl-triethoxydilane, 3-(trichlorosilyl)propyl methacrylate, and (3-bromopropyl)trimethoxysilane. Other non-limiting examples of compounds that may be used to form the linker include poly-lysine, collagen, fibronectin, and laminin.

In addition, in various embodiments, a wire may be prepared for binding or coating of a suitable biological effector by activating the surface of the wire, silanizing at least a portion of the wire, and reacting a crosslinker to the silanized portions of the wire. Methods for activating the surface include, but are not limited to, surface oxidation, such as by plasma oxidation or acid oxidation. Non-limiting examples of suitable types of crosslinkers that are commercially available and known in the art include maleimides, histidines, haloacetyls, and pyridyldithiols.

Similarly, the interaction between the linker and the molecule to be delivered can be covalent, electrostatic, photosensitive, or hydrolysable. In some embodiments, a molecule of interest attached to or coated on a wire may be a biological effector. As used herein, a “biological effector” refers to a substance that is able to modulate the expression or activity of a cellular target. It includes, but is not limited to, a small molecule, a protein (e.g., a natural protein or a fusion protein), an enzyme, an antibody (e.g., a monoclonal antibody), a nucleic acid (e.g., DNA, including linear and plasmid DNAs; RNA, including mRNA, siRNA, and microRNA), and a carbohydrate. The term “small molecule” refers to any molecule with a molecular weight below 1000 Da. Non-limiting examples of molecules that may be considered to be small molecules include synthetic compounds, drug molecules, oligosaccharides, oligonucleotides, and peptides. The term “cellular target” refers to any component of a cell. Non-limiting examples of cellular targets include DNA, RNA, a protein, an organelle, a lipid, or the cytoskeleton of a cell. Other examples include the lysosome, mitochondria, ribosome, nucleus, or the cell membrane.

In some cases, the wires can be used to deliver biological effectors or other suitable biomolecular cargo into a population of cells at surprisingly high efficiencies. Furthermore, such efficiencies may be achieved regardless of cell type, as the primary mode of interaction between the wires and the cells is physical insertion, rather than biochemical interactions (e.g., as would appear in traditional pathways such as phagocytosis, receptor-mediated endocytosis, etc.). For instance, in a population of cells on the surface of the substrate, at least about 50%, at least about 60%, at least about 70%, at least about 80%, or at least about 90% of the cells may have at least one wire inserted therein. In some cases, as discussed herein, the wires may have at least partially coated thereon one or more biological effectors. Thus, in some embodiments, biological effectors may be delivered to at least about 50%, at least about 60%, at least about 70%, at least about 80%, or at least about 90% of the cells on the substrate, e.g., via the wires.

In one set of embodiments, the surface of the substrate may be treated in any fashion that allows binding of cells to occur thereto. For example, the surface may be ionized and/or coated with any of a wide variety of hydrophilic and/or cytophilic materials, for example, materials having exposed carboxylic acid, alcohol, and/or amino groups. In another set of embodiments, the surface of the substrate may be reacted in such a manner as to produce carboxylic acid, alcohol, and/or amino groups on the surface. In some cases, the surface of the substrate may be coated with a biological material that promotes adhesion or binding of cells, for example, materials such as fibronectin, laminin, vitronectin, albumin, collagen, or peptides or proteins containing RGD sequences.

It should be understood that for a cell to adhere to the wire, a separate chemical or “glue” is not necessarily required. In some cases, sufficient wires may be inserted into a cell such that the cell cannot easily be removed from the wires (e.g., through random or ambient vibrations), and thus, the wires are able to remain inserted into the cells. In some cases, the cells cannot be readily removed via application of an external fluid after the wires have been inserted into the cells.

In some cases, merely placing or plating the cells on the wires is sufficient to cause at least some of the wires to be inserted into the cells. For example, a population of cells suspended in media may be added to the surface of the substrate containing the wires, and as the cells settle from being suspended in the media to the surface of the substrate, at least some of the cells may encounter wires, which may (at least in some cases) become inserted into the cells.

Any suitable type of cell may be used. For example, the cell may be a prokaryotic cell or a eukaryotic cell. The cell may be from a single-celled organism or a multi-celled organism. In some cases, the cell is genetically engineered, e.g., the cell may be a chimeric cell. The cell may be bacteria, fungi, a plant cell, an animal cell, etc. The cell may be from a human or a non-human animal or mammal. For instance, if the cell is from an animal, the cell may be a cardiac cell, a fibroblast, a keratinocyte, a hepatocyte, a chondrocyte, a neural cell, an osteocyte, an osteoblast, a muscle cell, a blood cell, an endothelial cell, an immune cell (e.g., a T-cell, a B-cell, a macrophage, a neutrophil, a basophil, a mast cell, an eosinophil), etc. In some cases, the cell is a cancer cell.

In some embodiments, the cells have protective outer layers (e.g. plant cells, such as plant pollen, spores, or the like). In certain cases the cells may be present in seeds or tissues. In some cases the outer layers could be native biological protections (e.g. complex exine for pollens), other types of cells, or general biological materials. Such plant cells are often difficult to penetrate with needles, e.g., due to their protective outer layers. Accordingly, in certain embodiments, the micro- and nanoneedles are sized to be able to penetrate such cells. For instance, the needles may comprise a first portion that having a first diameter, e.g., that is sufficient to prevent the needle from buckling or becoming distorted during penetration of a protective outer layer, and a second portion, e.g., having a second diameter, that actually penetrates a target, e.g., a plant stem cell or other cell such as described herein.

Thus, for instance, a variety of different cell types may be exposed to a common biological effector in certain embodiments, e.g., to determine the effect of the common biological effector on such cells. For example, the biological effector may be a small molecule, RNA, DNA, a peptide, a protein, or the like. As non-limiting examples, the cell types may be bacteria or other prokaryotes, and the common biological effector may be a suspected drug or antimicrobial agent. In some cases, at least 10, at least 20, at least 30, at least 40, at least 50, at least 60, at least 70, at least 80, at least 90, at least 100 cells, at least 500 cells, at least 1000 cells, at least 5000 cells, at least 10,000 cells, at least 50,000 cells, at least 100,000 cells, etc. may be studied. For example, the different cell types may each be placed into distinct wells of a multiwell plate, and wires inserted into the cells placed in each of the wells to insert a common biological effector.

In another set of embodiments, different common biological effectors may be studied, e.g., as applied to a single or clonal population of cells, or to a variety of different cell types such as those discussed above. For instance, the wells of a multiwell plate may contain wires, and at least some of the wires may be at least partially coated with a variety of biological effectors. For example, at least 10, at least 20, at least 30, at least 40, at least 50, at least 60, at least 70, at least 80, at least 90, at least 100, at least 500, at least 1000, at least 5000, at least 10,000, at least 50,000, at least 100,000, etc. different biological effectors may be studied. In some cases, the biological effectors may be added to the wells and the wires using robotic systems such as those discussed herein. Accordingly, cells placed in the wells of the multiwell plate may encounter different biological effectors, as inserted by the wires. As a non-limiting example, the different biological effectors may represent a plurality of suspected candidate drugs, and the effects of the various candidate drugs on a given population of cells may be studied to identify or screen drugs of interest.

In addition, it should be noted that in some embodiments, the cells may be cultured on the substrate using any suitable cell culturing technique, e.g., before or after insertion of wires. For example, mammalian cells may be cultured at 37° C. under appropriate relative humidities in the presence of appropriate cell media. Thus, for instance, the effect of a candidate drug (or a plurality of candidate drugs) on the effect of a suitable population of cells may be studied.

The following documents are incorporated herein by reference in their entireties: U.S. Pat. No. 9,304,132; U.S. Pat. Apl. Pub. No. 2013-0284612, 2013-0260467, 2015-0203348, and 2015-0191688; Int. Pat. Apl. Pub. No. WO 2016/112315; and U.S. Pat. Apl. Ser. Nos. 62/580,126 and 62/529,683.

U.S. Provisional Patent Application Ser. No. 62/596,533, filed Dec. 8, 2017, entitled “Micro- and Nanoneedles for Plant Cell Penetration,” by Park, et al., is also incorporated herein by reference in its entirety.

The following examples are intended to illustrate certain embodiments of the present invention, but do not exemplify the full scope of the invention.

### Example 1

This example illustrates biomolecule delivery at tunable penetration depths using tall nano- and micro-sized needles, including the delivery of siRNA into pollen grains by quartz nanoneedles.

Fluorescent dye labelled siRNA was used to demonstrate the delivery of molecules into pollen grains with additional pressure applied between a quartz needle array and pollen grains. Dandelion pollen were collected from flowers the same day of experiment and stirred in the culture medium to obtain a uniform suspension. The pollen suspension was kept standing in a plastic petri dish to allow a monolayer of pollen grains settled on the flat bottom surface of the dish.

Then, a cy3-siRNA coated quartz nanoneedle array was placed on top of the pollen monolayer with needles facing down. Gentle force was applied by tweezers on the back side of the needle array to press the needle array against the bottom of the dish, allowing the needles to penetrate into the seed and into the cell walls. After that, the needle array was picked up by tweezers and immense in clean medium with needles facing up. Pollen penetrated by the needles were taken together with the needle array and characterized 1 hour after the penetration with confocal microscope. The negative control was done by adding cy3-siRNA into culture medium and clean with fresh medium 1 hour later.

For SEM observation, the pollen was fixed in a 4% aqueous solution of paraformaldehyde followed by ethanol replacement and critical point drying. FIGS. 13A and 13B are SEM images showing that quartz needles successfully penetrate the out layers of the pollen grains with the whole grains still in good shape. The pollen penetrated by needle array shows significant higher fluorescent intensity compared to negative control ones, in FIGS. 13C and 13D. These fluorescent images were taken with the same laser and filter settings.

FIG. 13 shows the delivery of siRNA into pollen grains by quartz nanoneedle array. FIGS. 13A and 13B are SEM images of pollen grains penetrated by the quartz needles. FIGS. 13C and 13D are fluorescent microscope images showing the delivery of cy-3 siRNA into pollen grains. FIG. 13C is a negative control with dye labelled molecules in the medium without needle penetration. FIG. 13D shows pollen grains penetrated by needles with cy-3 siRNA loaded on the needles.

### Example 2

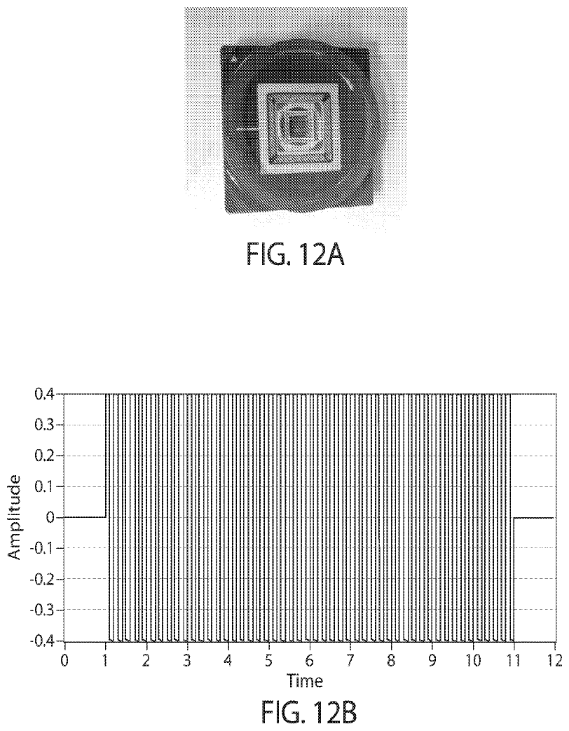

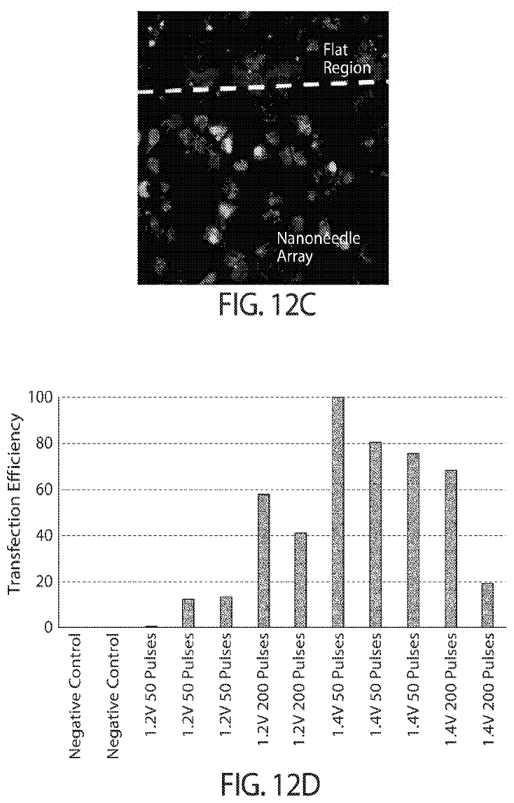

Applying an electrical bias to the very tip part of nano- and micro-sized needles helps to permeabilize proximate cell membranes, allowing biomolecules to diffuse across the barrier to the intracellular cytosol. In this example, to achieve this needle-based permeabilization, a layer of 30 nm Pt was sputtered on top of a fabricated quartz nanoneedle array with 10 nm Ti as adhesive layer to make the whole nanoneedle conductive. Photolithography and dry etching of Pt were utilized to define groups of needles and connect them to electrode pads, which later were wire-bonded to pins of a customized chip carrier. A layer of SiO₂, with typical thickness of 200 nm, was conformally deposited on the needle array by atomic layer deposition (ALD) to encapsulate the metal surface. The needle tip was then exposed by spin-coating a layer of polymer resist and selectively etching the ALD SiO₂ around the needle tip (FIG. 11). Finally, the needle array was packaged on a chip carrier and connected to external electronics (FIG. 12A). A typical device had 1 million functional needles with uniform area of exposed metal surfaces, connecting to 16 individually controllable pins of the chip carrier.

The electrical bias could be applied in the form of a voltage or a current. In voltage mode, a pulse sequence with predesigned amplitude, offset, pulse duration as well as pulse number was delivered to each needle group (FIG. 2B). The current flowing through each group was measured but used for monitoring purposes. In current mode, the voltage amplitude applied was controlled with proportional integral (PI) feedback of the measured current to set a desired current value. In this mode, the current was kept at a stable value, typically for ˜20 to 60 s. The 16 individual electrodes could be addressed individually or simultaneously, allowing the ability to quickly screen parameters.

To test the device, human embryonic kidney (HEK293) cells were cultured on top of the device and a GFP plasmid was utilized as a model molecule to be delivered. The GFP plasmid was added to the culture medium with final concentration of 10 micrograms/ml 1 hour ahead of the experiment. The electrical bias was applied in voltage mode to groups of nanoneedles with amplitudes ranging from 0.5 V to 1.6 V. The typical pulse duration was 100 ms and the pulse number ranged from 50 to 200 pulses. The cells were cultured at 37° C. for 48 hours before observation to allow the GFP plasmid to be expressed. The delivery efficiency was calculated by dividing the GFP expressing cells to the total number of cells observed. From the preliminary data, the HEK293 cells showed considerable GFP expression with amplitude of 1.2 V with 50 pulses. The transfection efficiency increased with the increase of both the amplitude and pulse number and reaches 85+/−13% (sample size 3) with 1.4 V amplitude and 50 pulses. Beyond 1.4 V and 200 pulses, less transfection efficiency was observed possibly due to poor cell viability. See FIGS. 12C and 12D.

FIG. 11 shows a quartz nanoneedle array with exposed metal tips. FIGS. 11A and 11B are SEM images of fabricated quartz nanoneedles. FIGS. 11C and 11D are BSE images of fabricated quartz nanoneedle, exposed Pt shows higher contrast compared to ALD SiO₂. All images are obtained with a 30° tilt.

FIG. 12A shows a packaged device with a quartz nanoneedle array mounted on a customized chip carrier. FIG. 12B shows a typical voltage signal pulse sequence applied to a group of nanoneedles for permeabilization. FIG. 12C is a confocal optical image showing the GFP transfection by delivering GFP plasmid into cells by the nanoneedle array. FIG. 12D shows GFP transfection efficiency vs. parameters of the applied pulse sequence.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
inserting an array of nanowires into a plant cell, wherein the wires are substantially vertically positioned on a substrate; and
delivering a biomolecule internally of cells within the plant cell via at least some of the nanowires.

2. The method of claim 1, wherein the plant cell is pollen.

3. The method of claim 1, wherein the plant cell is a spore.

4. The method of claim 1, wherein the plant cell comprises a coating having a thickness of at least 1 micrometer.

5. The method of claim 1, wherein the plant cell comprises an exine and/or an intine.

6. The method of claim 1, wherein the biomolecule comprises a nucleic acid.

7. The method of claim 1, wherein the biomolecule is attached to the nanowires via a linker.

8. The method of claim 7, wherein the linker comprises a silane.

9. The method of claim 1, wherein the biomolecule coats at least some of the nanowires.

10. The method of claim 1, wherein the array of nanowires and the substrate are formed of the same material.

11. The method of claim 1, wherein the nanowires comprises semiconductor nanowires.

12. The method of claim 1, wherein the nanowires comprises quartz nanowires.

13. The method of claim 1, wherein the nanowires comprises metal nanowires.

14. The method of claim 1, wherein at least some of the nanowires are electrically conductive.

15. The method of claim 1, wherein at least some of the nanowires have a coating.

16. The method of claim 15, wherein the coating is electrically conductive.

17. The method of claim 1, wherein the nanowires are oriented at an angle of at least 60° relative to the substrate.

18. The method of claim 1, wherein the nanowires are oriented at an angle of between 80° and 100° relative to the substrate.

19. The method of claim 1, comprising delivering a biomolecule internally to the plant cell.

20. The method of claim 1, wherein the biomolecule is delivered greater than 1 micrometer from the cell surface.

21. The method of claim 1, further comprising electroporating the cells to facilitate biomolecule delivery.

22. The method of claim 21, comprising applying electricity to at least some of the nanowires to cause electroporation.

23. The method of claim 1, wherein the nanowires has an average length of at least 10 micrometers.

24. The method of claim 1, wherein the nanowires has an average length of at least 50 micrometers.

25. The method of claim 1, wherein at least some of the nanowires comprising a first substantially cylindrical portion having a first diameter, and a second substantially cylindrical portion having a second diameter smaller than the first portion.

26. The method of claim 25, wherein the first portion has a length of at least 10 micrometers.

27. The method of claim 25, wherein the first portion has a length of at least 50 micrometers.

28. The method of claim 25, wherein the first portion has an average diameter of less than 300 nm.

29. The method of claim 25, wherein the second portion has a length of at least 10 micrometers.

30. The method of claim 25, wherein at least 70% of the length of at least some of the nanowires is the first portion.

31. An article, comprising:
an array of wires attached at a first end on a substrate, at least some of the nanowires comprising a first substantially cylindrical portion having a first diameter, and a second substantially cylindrical portion having a second diameter smaller than the first portion.

32. The article of claim 31, wherein the first portion has a length of at least 10 micrometers.

33. The article of claim 31, wherein the first portion has a length of at least 50 micrometers.

34. The article of claim 31, wherein the first portion has an average diameter of less than 300 nm.

35. The article of claim 31, wherein the second portion has a length of at least 10 micrometers.

36. The article of claim 31, wherein at least 70% of the length of at least some of the nanowires is the first portion.

37. The article of claim 31, wherein at least 90% of the length of at least some of the nanowires is the first portion.

38. The article of claim 31, wherein the array of nanowires and the substrate are formed of the same material.

39. The article of claim 31, wherein the nanowires comprises semiconductor nanowires.

40. The article of claim 31, wherein the nanowires comprises quartz nanowires.

41. The article of claim 31, wherein the nanowires comprises metal nanowires.

42. The article of claim 31, wherein at least some of the nanowires are electrically conductive.

43. The article of claim 31, wherein at least some of the nanowires have a coating.

44. The article of claim 43, wherein the coating is electrically conductive.

45. The article of claim 31, wherein the nanowires are oriented at an angle of between 80° and 100° relative to the substrate.

46. An article, comprising:
a plant cell; and
an array of nanowires inserted into the plant cell, wherein the wires are substantially vertically positioned on a substrate.

47. The article of claim 46, wherein the plant cell is pollen.

48. The article of claim 46, wherein the plant cell is a plant spore.

49. The article of claim 46, wherein the array of nanowires and the substrate are formed of the same material.

50. The article of claim 46, wherein the nanowires comprises semiconductor nanowires.

51. The article of claim 46, wherein the nanowires comprises quartz nanowires.

52. The article of claim 46, wherein the nanowires comprises metal nanowires.

53. The article of claim 46, wherein at least some of the nanowires are electrically conductive.

54. The article of claim 46, wherein at least some of the nanowires have a coating.

55. The article of claim 54, wherein the coating is electrically conductive.

56. The article of claim 46, wherein the nanowires are oriented at an angle of at least 60° relative to the substrate.

57. The article of claim 46, wherein the nanowires are oriented at an angle of between 80° and 100° relative to the substrate.

58. A method, comprising:
inserting an array of nanowires into a plant cell, wherein the wires are substantially vertically positioned on a substrate; and
delivering a biomolecule internally of the cell via at least some of the nanowires.

59. The method of claim 58, wherein the plant cell is pollen.

60. The method of claim 58, wherein the plant cell is a spore.

61. The method of claim 58, wherein the plant cell comprises a coating having a thickness of at least 1 micrometer.

62. The method of claim 58, wherein the plant cell comprises an exine and/or an intine.

63. The method of claim 58, wherein the biomolecule comprises a nucleic acid.

64. An article, comprising:
an array of wires attached at a first end on a substrate, at least some of the nanowires comprising a first portion having a first diameter, and a second portion having a second diameter smaller than the first portion.

65. The article of claim 64, wherein the first portion is closer to the surface than the second portion.

66. The article of claim 64, wherein the first portion comprises at least 50% by length of the nanowire.

67. The article of claim 64, wherein the first portion comprises at least 90% by length of the nanowire.

68. The article of claim 64, wherein the wires comprise microneedles.

69. The article of claim 64, wherein the wires comprise nanoneedles.

70. An article, comprising:
a plant cell;
one or more wires having a first end inserted into the plant cell; and
a substrate that the one or more wires is attached to via a second end.

71. The article of claim 70, wherein the wires comprise microneedles.

72. The article of claim 70, wherein the wires comprise nanoneedles.

73. A method, comprising:
inserting an array of wires into a plant cell, wherein at least some of the wires are attached to a surface at a first end and comprise a first portion having a first diameter, and a second portion having a second diameter smaller than the first portion

74. The method of claim 73, wherein the wires comprise microneedles.

75. The method of claim 73, wherein the wires comprise nanoneedles.
